(19) United States
(12) Patent Application Publication
(10) Pub. No.: US 2026/0115273 A1
(43) Pub. Date: Apr. 30, 2026

(54) VIRUS ATTENUATION

(71) Applicant: University of Lancaster, Lancaster (GB)

(72) Inventor: Muhammad MUNIR, Lancaster (GB)

(21) Appl. No.: 18/572,367

(22) PCT Filed: Jun. 21, 2022
(86) PCT No.: PCT/GB2022/051579

§ 371 Date: Dec. 20, 2023

(30) Foreign Application Priority Data
Jun. 23, 2021 (GB) 2108986.7
Dec. 21, 2021 (GB) 2118670.5

(51) Int. Cl. A61K 39/155; A61K 39/00; A61P 37/04

(52) U.S. Cl. CPC A61K 39/155; A61P 37/04; A61K 2039/5254; C12N 2760/18021; C12N 2760/18034; C12N 2760/18062; C12N 2760/18071

(57) ABSTRACT

The present disclosure relates to paramyxoviruses, in particular attenuated avian avulaviruses (para, ortho and meta), mutated and genetically modified forms, as well as a vaccine formulation comprising an attenuated avian avulavirus and uses/methods of use thereof.

### FIELD OF THE DISCLOSURE

The present disclosure relates to paramyxoviruses, in particular attenuated avian avulaviruses (para, ortho and meta), mutated and genetically modified forms, as well as a vaccine formulation comprising an attenuated avian avulavirus and uses/methods of use thereof.

### BACKGROUND TO THE DISCLOSURE

Avian orthoavulaviruses (AOaV; formerly known as avian paramyxoviruses) infect nearly every order of birds worldwide and are responsible for a significant mortality and economic losses in the poultry industry. The severity of the disease varies from no clinical signs (asymptomatic) infection to a highly pathogenic disease, and this depends upon multiple factors of host and virus origin. These are found in a diverse range of wild birds, particularly those associated with water.

According to the level of disease severity in chicken, AOaV are classified into following pathogenic types (pathotypes): (1) Lentogenic (low or apathogenic): the AOaV strains that cause slight illness or asymptomatic respiratory infection. (2) Mesogenic: AOaV strains showing low mortality, but acute respiratory and neurological symptoms in some of the poultry. (3) Velogenic (high-pathogenic) AOaV strains that cause high mortality (up to 100%) and multiorgan lesions in the poultry. The velogenic AoaV strains are further divided into: (a) viscerotropic velogenic AOaV: these AOaV strains target the digestive system organ and cause lesions and high mortality, (b) neurotropic velogenic AOaV: the AOaV strains that mainly cause respiratory and neurological symptom, and high mortality.

Based on the latest nomenclature, the avian meta-, para- and orthoavulaviruses belong to the subfamily Avulavirinae of Paramyxoviridae family, are enveloped, and single-stranded viruses carrying negative sense RNA (ss-ve) of approximately 15186 nucleotides (Krishnamurthy & Samal, 1998: Phillips et el. 1998; de Leeuw & Peeters, 1999). There are over 20 species of avian ortho-, meta-, and para-avulaviruses described by the International Committee on Taxonomy of Viruses (ICTV). Among all these species, AOaV serotype 1 (AOaV-1 are the most important viruses due to their disease-causing capabilities in commercial poultry around the world). The AOaV-1 strains are further divided into classes I and II, each containing defined genotypes and sub-genotypes. Avirulent genotypes as well as the virulent genotypes that cause disease are classified within class II, whereas class I contains only avirulent viruses and is commonly found in waterfowl and shorebirds.

Reverse-genetic techniques for the generation of infectious virus particles from cloned cDNA have been reported previously (Conzelmann, 1996) and several reverse-genetic technologies have been proposed for AOaVs (Romer-Oberdorfer et al., 1999; Peeters et al., 1999), the contents of which are hereby incorporated by way of reference. However, all available AOaVs-based live attenuated and recombinant vaccines are based on avirulent AOaVs namely LaSota strains. Owing to enhanced pathogenicity of velogenic strains, these cannot be used as live attenuated vaccines and such vaccine use requires attenuation. Conventionally, the attenuation of viruses is made through: (i) serial passaging of pathogenic viruses in either cell culture, eggs or in vivo. The randomly acquired mutations can render a pathogenic virus to apathogenic, which are then exploited as vaccine candidate or vaccine vector; (ii) modification of virulent factors of the virus allow attenuation; (iii) modification of the cleavage sites or viral genes to restrict the host tropism. Therefore, currently, AOaV strains being used as vaccine vectors are based on either lentogenic strains or attenuated strains (by changing the F0 cleavage site, of the F protein, which changes a velogenic strain to lentogenic strain). The small modifications, in latter case, are likely to revert into velogenic and may pose a risk for enhanced virulence; or (iv) modification of the genetic coding of multiple genes (HN, F and P, for example) to convert mesogenic strains, which are low pathogenic in nature, into attenuated strains (Wang et al., 2019). However, mesogenic strains have limited tropism to multiple organs and are therefore likely to be insufficient in induction of systemic immune responses. Therefore, there is a need to produce novel vaccine vectors, which are safe, effective, immunogenic and can be multipurposed.

Newcastle disease virus (NDV) is a representative AOaV and attenuated strains of NDV have been used in cancer therapies, for example.

### SUMMARY OF THE DISCLOSURE

In a first teaching, there is provided an attenuated velogenic avian orthoavulavirus (AOaV), wherein each of the HN and F genes of an AOaV genome of the attenuated AOaV comprises, consists essentially of, or consists of a plurality of silent mutations, as compared to a wild-type or parent velogenic AOaV from which the attenuated AOaV has been derived.

The skilled reader understands that silent mutations are mutations in the genome that do not alter the amino acid sequence of the HN and F genes when translated. Typically, this is achieved through the use of codon deoptimisation. Each of the HN and F genes includes a plurality of silent mutations. Typically, each of the HN and F genes includes at least 5, 10, 15, 20, 25, 30, 40, 50 or more mutated codons. A significant number of silent mutations are generally introduced, in order to minimise the possibility of a mutated attenuated strain reverting back to a velogenic strain. Typically, the proportion of identical codons in the HN and/or F genes, as compared to the wild-type or parent strain, from which the attenuated AOaV has been derived, may be less than 95%, 90%, 85%, 80%, 75%, 70%, 60% or 50%. For example, only 1%, 2%, 5%, 10%, 20%, 25%, 30%, 40% of wild-type or parent strain codons, may remain unmutated in the attenuated strain. Desirably, mutations occur across the entire gene sequences, including the N-terminus, middle region and C-terminus of each HN and F gene.

The current teaching is generally attributed to the attenuation of velogenic AOaV viruses into avirulent viruses using “codon deoptimization” which refers to synonymous mutation of the viral protein to use rare codons in the genetic backbone of AOaV, which may (a) lead to the reduction in expression of the viral genes (b) to attenuate the velogenic AOaV viruses without changing the viral protein sequences, (c) changes in the structure of the viral genome leading to differential identification and transcription (d) enchance innate immunity due to introduced RNA structures, and/or (d) making non-revertant viruses due to high proportion of the genetic mutations. By “rare codons” is meant that a codon found in the wild-type or parent codon, is swapped for a less favoured synonymous codon, as used by the particular AOaV. The skilled reader is well aware of the codons which encode the various amino acids and by looking at a particular AOaV genome, is able to easily ascertain which codons are more favoured than others.

A conventional codon deoptimization strategy, relies only on the use of least used codon. In order to establish and validate a novel suboptimal codon usage strategy in developing live attenuated vaccines, in one embodiment, an all-inclusive methodology was applied in codon deoptimizing an AOaV genome collectively called a “smart codon usage algorithm”.

Specifically, building from the Homo sapiens (human) genome assembly of GRCh38.p13, a codon usage database was outlined, and the following principles were incorporated:
1. Exploiting the fact that one amino acid is encoded by more one nucleotide triplet (referred here as synonymous codons) and that codon usage is at a variable frequency, codon which were used lower than that of normal human genes were considered without changing the amino acid sequence. This alteration reduces the adaptation of virus codon usage for host tRNA abundance and thus reduces the viral gene translation and fitness.
2. Based on the codon usage database, it's evident that different codon pairs carry variable frequency. Incorporation of this codon pair bias in the AOaV genomes affects translation of the viral genes due to predicted compatibility of codon pair bias in the translating ribosome RNA.
3. Incorporation of codon deoptimization and codon pair biasness allowed consideration of CpG and UpA contents in the AOaV genome to stimulate the immune system. This stimulates host innate immunity which has a negative impact on intra-host virus replication and fitness.
4. Incorporation of a high level of synonymous codons in amino acids, which represent high codon redundancy (leucine and serine), allowed lesser extent of evolutionary potential in the AOaV genome, yielding fewer fit viruses.
5. Codon deoptimization adversely affects RNA secondary structure and can impact folding of RNA which limited the replication fitness of the AOaV. During codon deoptimization, a folding free energy was considered to ensure the rescuing potential of the virus while maintaining the usual amino acid sequence of the virus and changing the codon pair bias.
6. During codon deoptimization, less favourable features for virus replication kinetics including RNA hairpins, large secondary structures, and stem loop of AOaV genomes were incorporated.

In one embodiment, a combination of at least 3 of the above principles is employed. For example, 1 and 2 may be employed in combination with at least one, two or three of 3-6 or 4-6.

An algorithm was designed to incorporate codon optimization strategy for each of 3721 possible codon pairs using Smart Codon Usage Algorithm (SCUA):

$$SCUA = \frac{\frac{CfOt}{CfOr} NOt}{NOr} \times vgf$$

Here,
CfOt: frequency of codon occurrence in test sequence
CfOr: frequency of codon occurrence in reference sequence
NOt: number of codon occurrences in test sequence
NOr: number of codon occurrences in reference sequence
vgf: viral genomic features.

The term “attenuated AOaV” as used herein is understood to refer to an AOaV, which has a reduced virulence as compared to the velogenic wild-type or parent virus from which it has been derived, but which is still viable (or “live”). Attenuation takes a velogenic AOaV and alters it so that it becomes harmless or less virulent. Virulence can be determined by techniques known in the art, such as the ability to infect a host; time taken to kill a host; and/or intracerebral pathogenicity index, for example. Velogenic strains are distinguished from Lentogenic and mesogenic strains. Velogenic strains most commonly cause severe disease in birds, such as chickens, with mortality; signs principally respiratory and/or nervous. Initial clinical signs vary but include: lethargy, inappetence, ruffled feathers, oedema and injection of conjunctiva. As the disease progresses birds may develop: greenish or white watery diarrhoea, dyspnoea and inflammation of the head and neck often with cyanotic discoloration. In line with recognised WHO for animal health teaching (OIE Terrestrial Manual 2000, Appendix 3), a velogenic avian orthoavulavirus may be considered in a mean death time in eggs (MDT) test to be less than 60 hours. Alternatively in an Intracerebral pathogenicity index (ICPI) test in day-old chicks, velogenic viruses have an ICPI of higher than 1.4.

As well as the attenuated AOaVs as described herein, this teaching also provides a modified AOaV genome, which is capable of generating said attenuated AOaVs. A modified AOaV genome as described herein, may also be considered as being a vector, for the delivery of one or more agents, which are exogenous to the specific AOaV. Thus, for example, a modified AOaV genome may be further modified in order to express one or more proteins or antigenic fragments thereof, from another virus. In this manner, upon expression of the modified AOaV genome, the attenuated AOaV so produced, also expresses said one or more proteins or antigenic fragment from a different virus, so that a host may induce an immune response to the AOaV and also said one or more proteins or antigenic fragment from the different virus.

In accordance with the teaching herein, it has been observed that replacement of one or more natural (or native) codons in an AOaV with a synonymous unpreferred (deoptimised) codon(s) and structural changes in the viral RNA can decrease the virulence of the AOaV, thereby attenuating the AOaV. The unpreferred synonymous codon(s) encode the same amino acid as the native codon(s) but have nonetheless been found to reduce a pathogen's virulence. The introduction of multiple deoptimized codons into the HN and F genes of an AOaV can limit the ability of the AOaV to mutate or to use recombination to become virulent. The disclosed compositions and methods can be used in attenuated vaccines having recued virulence, or substantially no disease causing ability and optionally enhanced genetic stabilities.

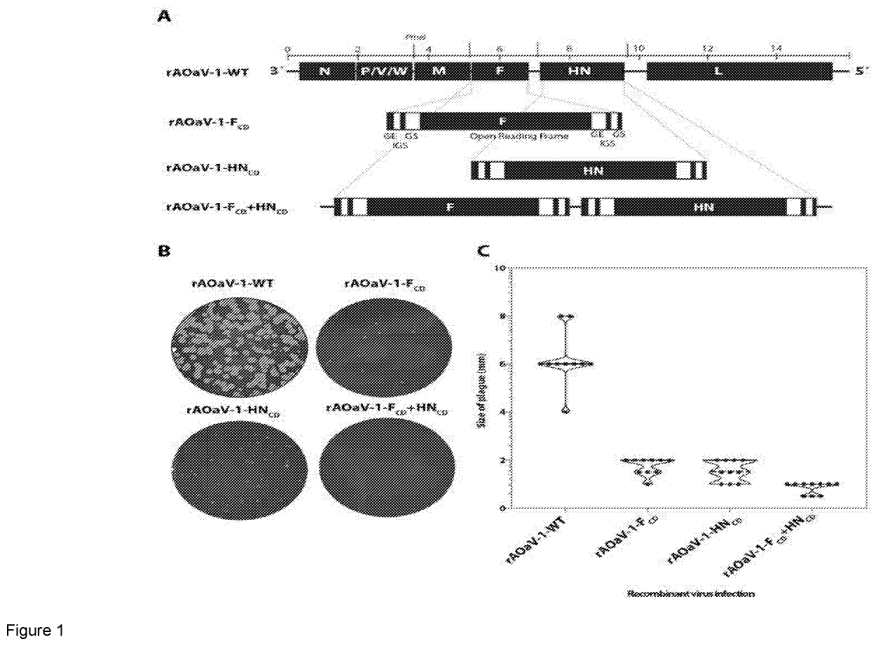

The genome of AOaVs consists of six linear genes that are transcribed by viral polymerase through promoter region at the 3′ end of the genome using a sequential stop-start mechanism. All transcribed genes encode for a single protein, in the order of nucleocapsid protein (NP), phosphoprotein (P), matrix protein (M), fusion protein (F), haemagglutinin-neuraminidase (HN) and large polymerase protein (L) except the P gene. The P gene translate into additional two non-structural proteins (V and W) through a mechanism called RNA editing (Steward et al., 1993). A schematic diagram of the genomic organization of AOaVs is shown in FIG. 1A. Each gene of AOaVs is flanked by short transcription signal called the gene-start (GS) signal at the 3′ end of the gene followed by open reading frame (ORF) and transcription stop signal called gene-end (GE) at the 5′ end of the gene. The GE is also involved in the synthesis of polyA tail in the transcribed mRNA. All genes are then separated by a stretch of sequence called intergenic sequence (IGS).

The NP, P and L proteins are major components of replication machinery where NP proteins surround the genome, and P and L form the functional nucleocapsid for the viral transcription and replication. The surface of AOaV is decorated by two glycoproteins called HN and F proteins. The HN allows the virus to bind to a host, and the F protein allows the virus to fuse with the host cell. Biochemically, the F protein is a type I membrane glycoprotein and forms a trimeric structure (trimer). The F protein is made as a non-active precursor form (F0) and is divided into the di-sulfide linked subunits F1 and F2 when the precursor F0 molecule passes through Golgi membranes. In order for the AOaV to infect a cell, it is necessary for the precursor glycoprotein F0 to be cleaved into F1 and F2. This post-translational cleavage is intervened by proteases of a host cell. If the cleavage does not occur, non-infectious virions are generated, and the virus replication cannot progress. The F0 protein of a velogenic AOaV viruses can be cleaved by various proteases therefore these cause a fatal systemic infection, but the F0 protein of a lentogenic virus, for example, is restricted to respiratory organs or intestinal tract. The HN protein belongs to the type II membrane glycoprotein and forms a tetramer on the surface of the viral envelope, to penetrate into a cell membrane. The HN protein causes the virion to locate on the host cell surface via binding to sialic acids of glycoconjugates. The HN protein is divided into the three regions of a transmembrane domain, a stalk domain and a globular domain. Both a binding site of an antigenic receptor and an active site of neuraminidase locate on the globular domain. Both the HN and F proteins are primary target for the immune responses and most of neutralizing epitopes are mapped within these two surface glycoproteins.

As well as the codon alterations described herein, in relation to the HN and F genes, optionally one or more of the other proteins encoded by the AOaV genome, may include one or a plurality of silent mutations. In one embodiment, codon alterations which have been specifically introduced (to distinguish from naturally occurring ones), have only been introduced into the HN and F genes of the AOaV genome, such as a velogenic AOaV genome.

As well as the use of silent mutations, such as through codon deoptimized methods described herein, the AOaVs described can also incorporate mutations into the amino acid sequence that are either derived from the parent gene sequence, are known to exist for the gene or protein encoded by the gene, or occur in the deoptimized gene de novo during the lifecycle of a virus having the deoptimized gene. In some embodiments the mutation can be a coding mutation, giving rise to a different amino acid residue in a given protein. In other embodiments, the mutation may occur in a gene having an unmodified, or parental sequence.

The live attenuated AOaVs reported herein are generated by incorporating nucleotide changes in the viral genome that deviate from natural codons which are found in a wild-type or parent strain and are incompetently translated by the host cell machinery. The introduced silent mutations do not alter protein sequences, which are originally encoded by the wild-type or parent virus. This allows the generation of same viral genetic background and antigenic features as that of wild type/parent virus strains, but alters the virulence of the attenuated virus.

The mutations, including silent mutations, described herein may be introduced into the genome of the AOaV using recombinant techniques well known to the skilled reader, such as described in Sambrook & Russell (Molecular Cloning: A laboratory Manual, Volume 2, (2001) CSHL Laboratory press), Alternatively, mutated HN and F gene sequences may be incorporated through de novo in vitro gene synthesis methods. Many companies offer such services including Thermo Fisher Scientific, Integrated DNA technologies, Eurofins and Genescript, for example.

Recombinant AOaVs in accordance with the present disclosure may be obtained through reverse genetic techniques known to the skilled reader in which a recombinant AOaV is obtained through use of 6 separate gene constructs encoding each of the 6 proteins of AOaV. Such a reverse genetic technique is described in Ayllon et al (2013), for example and further hereinafter.

The present disclosure further provides recombinant attenuated AOaVs suitable for use as a vaccine in a human or animal, such as a bird, and as an oncolytic and gene delivery platform. Thus, in a further teaching, there is provided a vaccine formulation comprising an attenuated AOaV or AOaV genome, as described herein. The vaccine, may optionally include one or more pharmaceutically acceptable excipients therefor and/or a suitable adjuvant as known in the art. A vaccine formulation may comprise a single or multiple attenuated AOaV strains. Moreover, a vaccine formulation may include attenuated viruses designed to protect a host against other viruses. It is typical in the art of vaccine development to provide such multivalent or multipathogen vaccines

The term “wild-type” as used herein refers to the phenotype of a virus, which is replication competent in susceptible animal and human hosts and may cause clinical disease. Derivative recombinant viruses with mutations but no substantial reduction in the replication fitness have the wild type phenotype. On the other hand, the viral derivatives that exhibit reduced viral replication in any assessment criteria can be considered restricted. Commonly, restricted replication of the virus in permissive host is associated with attenuation and lower disease severity. Therefore, attenuated virus infection in susceptible hosts will have reduced disease severity compared to wild-type version of the virus.

A “parent” virus refers to a virus that is used to derive the recombinant AOaVs of the present disclosure. That is, a virus prior to carrying out the mutations described herein.

The term gene sequence, gene(s) and genome refer to the sequence of the virus that encode a protein, polyprotein or open reading frames for such polyproteins.

Described herein are AOaVs, which are constituted by multiple codon carrying silent nucleotides and are introduced in multiple sites in the HN and F genes. Owing to the myriad of mutations in the HN and F genes, the attenuated recombinant viruses show stability and are suitable as live vaccine candidates against AOaVs infection.

The described deoptimized AOaVs nucleotide sequences can be used in combination with one or more than one deoptimized viral proteins. In some embodiments, the presented mutant viruses include one codon deoptimized gene or more than one gene. Therefore, such recombinant viruses can be produced by using the multiple genes, individually or in combination, provided herein.

The codon deoptimized mutant viruses described herein can be produced by incorporating mutations derived from parent gene sequences or by the one known to exist due to continuous replication of the virus in vitro, in ovo or in vivo.

The ability to engineer infectious AOaVs from cloned cDNA allows the incorporation of desired changes including but not limited to known mutations to attenuate the viruses, including gene deletion or modification. These incorporated changes may or may not represent biological features of the other virus strains. Exemplified herein are infectious AOaVs, which were produced by a synthetic approach. The infectious AOaVs are produced by the intracellular co-expression of cDNA that encode the AOaV's viral genome along with viral proteins essential to initiate transcription, replication and packaging of viruses. The method of producing the recombinant AOaVs described herein is disclosed along with a method to induce immune response in animals, mammals or human. The disclosure allows the incorporation of biologically driven mutations as well as additional mutations in recombinant AOaV vaccine candidate strains. The mutations that are deoptimized in the presented AOaVs can be used reversibly into the vaccine strains to regain the pathogenicity.

The attenuated AOaVs and their associated genomes disclosed herein may be selected as vaccine candidates based on replication efficacy, attenuation in ovo, immunogenicity, protection and/or phenotypic stability. The stability of candidate viruses is of importance in order to display an attenuated phenotype and replication in immune-competent hosts. Attenuated viruses described herein typically show improved stability, safety and/or immune induction compared to wild-type or parental strains.

To propagate the AOaVs viruses for vaccine production and other uses, cell lines and embryonated chicken eggs can be used. AOaVs grow in a range of cell lines and animal models. A high level of replication and virus yield may be achieved through replication of AOaV mutant viruses in Vero cells and/or chicken embryonated eggs. Viruses with multiple infection load may be inoculated at several temperatures to yield best inoculum and time to produce viruses.

The AOaVs, attenuated herein can be tested and assessed in several in vitro, in ovo and in vivo models to demonstrate attenuation, phenotypic reversion and immunogenicity for vaccine use. The same can be tested for thermolabile nature in animal models.

The disclosure further teaches isolated, purified and infectious AOaVs for vaccine, oncolytic, gene delivery and immune induction in animal, mammals or human hosts.

The teaching herein offer recombinant AOaVs, which can be used directly in vaccine, oncolyses, gene delivery and immune induction in animal, mammals, or human hosts.

Upon inoculation of AOaVs into the animal through, for example, injection, aerosol, or intranasal routes induced immunity against the HN and F proteins of the AOaVs, which are the most immunogenic antigens is expected to occur. This vaccination process is intended provide protection and resistance to infectious field strains.

A host to which the attenuated AOaV, AOaV genome and/or vaccine is to be administered can be any susceptible animal, mammals or human. The suitable hosts include birds, humans, non-human primates, bovine, ovine, caprine, rodents (mice or cotton rats, etc), equine, swine and lagamorph. In one embodiment, the host is a bird, such as a farmed bird, including chickens, turkeys, ducks and geese, for example. Appropriately, the teaching herein provides approaches to generate vaccines for a variety of veterinary and human applications.

The attenuated AOaVs, AOaV genomes or vaccines as described herein may be administered to the susceptible hosts, which are, or may be, at risk of infection. A single effective dose, or to enhance the immune responses, multiple doses may be delivered.

The precise amount vaccine and time of immune induction can be dependent on multiple factors including the host, health and environment.

The attenuated AOaVs, AOaV genomes or vaccines as described herein are expected to elicit protection, which is mediated through immune induction in the respiratory system and may protect the host from pneumonia and/or other respiratory conditions. Therefore, in one embodiment, this teaching offers ideal intranasal immunization, such as by an aerosol, liquid or dried powder administration

The level of attenuation of codon deoptimized mutants of AOaVs can be determined by quantifying the titre of the virus present at the site of inoculation or through systemic screening.

The current teaching is further directed toward generation of a recombinant AOaVs to express one or more immune genes and their use as an adjuvant or immune stimulant.

The current teaching is further directed toward generation of a recombinant AOaVs to express genes and to be used as such as an oncolytic agent or carrier of antibodies, nanobodies, synthetic agents and the like.

The invention also guides the generation of thermostable recombinant vaccines and generation of cleavage site mutated equivalent vector for the delivery of genes.

Within the scope of the present teaching is the ability for an AOaV genome to acts as a gene carrier, so as to be able to carry one or more exogenous genes from, for example, one or more viruses e.g. influenza virus, SARS-COV-2, Coronaviruses, human respiratory syncytial virus, human immunodeficiency virus, hepatitis A virus, hepatitis B virus, hepatitis C virus, poliovirus, rabies virus, Hendra virus, Nipah virus, human parainfluenza 3 virus, measles virus, mumps virus, Ebola virus, Marburg virus, West Nile virus, Japanese encephalitis virus, Dengue virus, Hantavirus, Rift Valley fever virus, Lassa fever virus, herpes simplex virus and yellow fever virus.

The exogenous gene may also be designed to target other avian diseases and/or infections. Thus, in one teaching, the exogenous gene may encode for a heterologous protein obtained from for example Avian and human Influenza (A) (Hemagglutinin (H5 and H7) and Neuraminidase), Avian leukosis virus (ALV) (env protein (gp85)), Chicken anemia virus (CAV) (VP1+VP2), Marek's disease virus (MDV) (glycoprotein B (gB), gH), Infectious laringotracheitis virus (ILT) (gB, gH, gD), Infectious bursal disease virus (IBDV) (VP2 and VP3), Turkey rhinotracheitis virus (TRT) (fusion (F) protein), Avian paramyxovirus-2, -3, -6 (PMV) (F-protein, Hemagglutinin neuraminidase (HN), or others, Infectious bronchitis virus (IBV) (peplomer protein, nucleoprotein), Reoviruses (sigma protein), Adenoviruses, Pneumoviruses, Salmonella enteritidis, Campylobacter jejuni, Escherichia coli, Bordetella avium (formerly Alcaligenes faecalis), Haemphilus paragallinarum, Pasteurella multocida, Ornithobacterium rhinotracheale, Riemerella (formerly Pasteurella) anatipestifer, Mycoplasmata (M. gallisepticum, M. synoviae, M. mereagridis, M. iowae), or Aspergilli (A. flavus, A. fumigatus).

The attenuated AOaVs, AOaV genomes as described herein may be formulated and administered, according to known methods, as a vaccine to induce an immune response in an animal, e.g., a mammal. Methods are well-known in the art for determining whether such attenuated vaccines have maintained similar antigenicity to that of a wild-type or parental strain derived therefrom.

Vaccine compositions of the present disclosure, suitable for inoculation or for parenteral, nasal or oral administration, comprise the attenuated AOaV or AoaV genome as described, optionally further comprising sterile aqueous or non-aqueous solutions, suspensions, and emulsions. The compositions can further comprise auxiliary agents or excipients, as known in the art. Such compositions may generally be presented in the form of individual doses (unit doses).

Preparations for parenteral administration include sterile aqueous or non-aqueous solutions, suspensions, and/or emulsions, which may contain auxiliary agents or excipients known in the art. Examples of non-aqueous solvents are propylene glycol, polyethylene glycol, vegetable oils such as olive oil, and injectable organic esters such as ethyl oleate. Carriers or occlusive dressings can be used to increase skin permeability and enhance antigen absorption. Liquid dosage forms for oral administration may generally comprise a liposome solution containing the liquid dosage form. Suitable forms for suspending liposomes include emulsions, suspensions, solutions, syrups, and elixirs containing inert diluents commonly used in the art, such as purified water. Besides the inert diluents, such compositions can also include adjuvants, wetting agents, emulsifying and suspending agents, or sweetening, flavoring, or perfuming agents.

When a composition of the present invention is used for administration to an individual, it can further comprise salts, buffers, adjuvants, or other substances, which are desirable for improving the efficacy of the composition. For vaccines, adjuvants, which can augment a specific immune response, can be used. Normally, the adjuvant and the composition are mixed prior to presentation to the immune system, or presented separately, but into the same site of the organism being immunized. Examples of materials suitable for use in vaccine compositions are described for example by the CDC in the US (see https://www.cdc.gov/vaccinesafety/concerns/adjuvants.html).

A vaccine composition according to the present teaching may further or additionally comprise at least one chemotherapeutic compound, for example, for gene therapy, immunosuppressants, anti-inflammatory agents or immune enhancers, and for vaccines, chemotherapeutics including, but not limited to, gamma globulin, amantadine, guanidine, hydroxybenzimidazole, interferon-α, interferon-β, interferon-γ, tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

The administration of the attenuated AOaVs, AOaV genomes, or vaccine compositions as described herein (or the antisera that it elicits) may be for either a “prophylactic” or “therapeutic” purpose. When provided prophylactically, the compositions, are provided before any symptom of a pathogen infection becomes manifest. The prophylactic administration of the composition serves to prevent or attenuate any subsequent infection. When provided prophylactically, the gene therapy compositions as described, are provided before any symptom of a disease becomes manifest. The prophylactic administration of the composition serves to prevent or attenuate one or more symptoms associated with the disease.

When provided therapeutically, a composition is provided upon the detection of a symptom of actual infection. The therapeutic administration of the composition serves to attenuate any actual infection. When provided therapeutically, a gene therapy composition is provided upon the detection of a symptom or indication of the disease. The therapeutic administration of the composition serves to attenuate a symptom or indication of that disease.

A composition of the present disclosure is physiologically significant if its presence results in a detectable change in the physiology of a recipient patient, e.g., enhances at least one primary or secondary humoral or cellular immune response against at least one strain of an AOaV. Only live vaccines administered by the respiratory route stimulate antibody in all mucosal surfaces as well as in serum. Thus, in one teaching, the present disclosure provides a live vaccine which is capable of presenting viral antigen to the upper respiratory tract to induce both local and systemic immunity. Small droplets penetrate into the lower respiratory tract thereby provoking a mainly humoral immune response, while coarse droplets stimulate local immunity in the upper respiratory tract. Therefore, aerosols with a wide range of droplet sizes generate the best overall local and humoral immunity.

The “protection” provided need not be absolute, i.e., the AOaV infection need not be totally prevented or eradicated, if there is a statistically significant improvement compared with a control population or set of patients. Protection may be limited to mitigating the severity or rapidity of onset of symptoms of the influenza virus infection.

An attenuated AOaV, AOaV genome, or vaccine composition according to the present disclosure may be administered by any means that achieve the intended purposes. For example, administration of such a composition may be by various parenteral routes such as subcutaneous, intravenous, intradermal, intramuscular, intraperitoneal, intranasal, oral or transdermal routes. Parenteral administration can be by bolus injection or by gradual perfusion over time.

A typical regimen for preventing, suppressing, or treating an AOaV related pathology, comprises administration of an effective amount of an attenuated AOaV, AOaV genome, or vaccine composition as described herein, administered as a single treatment, or repeated as enhancing or booster dosages, over a period up to and including between one week and about 24 months, or any range or value therein.

According to the present disclosure, an “effective amount” of a composition is one that is sufficient to achieve a desired biological effect. It is understood that the effective dosage will be dependent upon the age, sex, health, and weight of the recipient, kind of concurrent treatment, if any, frequency of treatment, and the nature of the effect wanted. The ranges of effective closes provided below are not intended to limit the invention and represent exemplary dose ranges. However, the dosage will be tailored to the individual subject, as is understood and determinable by one of skill in the art.

The dosage of an attenuated virus vaccine for a mammalian (e.g., human) or avian adult organism can be from about 10³-10⁷ plaque forming units (PFU)/kg, or any range or value therein. However, the dosage should be a safe and effective amount as determined by conventional methods, using existing vaccines as a starting point.

### DETAILED DESCRIPTION OF THE DISCLOSURE

The present disclosure will now be further described by way of non-limiting example and with reference to the figures, which show:

FIG. 1. A-Schematic representation of wild type (wt) and codon optimized (CD) AOaV-1. (A) rAOaV-1-WT contains genes in the order from N, P, M, F, HN and L. The rAOaV-1-F_{CD} contain full-length open reading frame of F which is codon deoptimized while rest of all genes carry sequence of rAOaV-1-WT. Similarly, the codon deoptimized HN gene replaced the corresponding WT in the construct rAOaV-1-HN_{CD}. The rAOaV-1-F_{CD}+HN_{CD} contains both HN and F gene codon deoptimized while rest of all genes remained unmodified except that the F protein was modified at the cleavage site; B—plaque assays showing the replication capacity of wild-type and mutated rAOaV viruses described herein; and C—graph showing the quantitative measurement of the plaques in FIG. 1B.

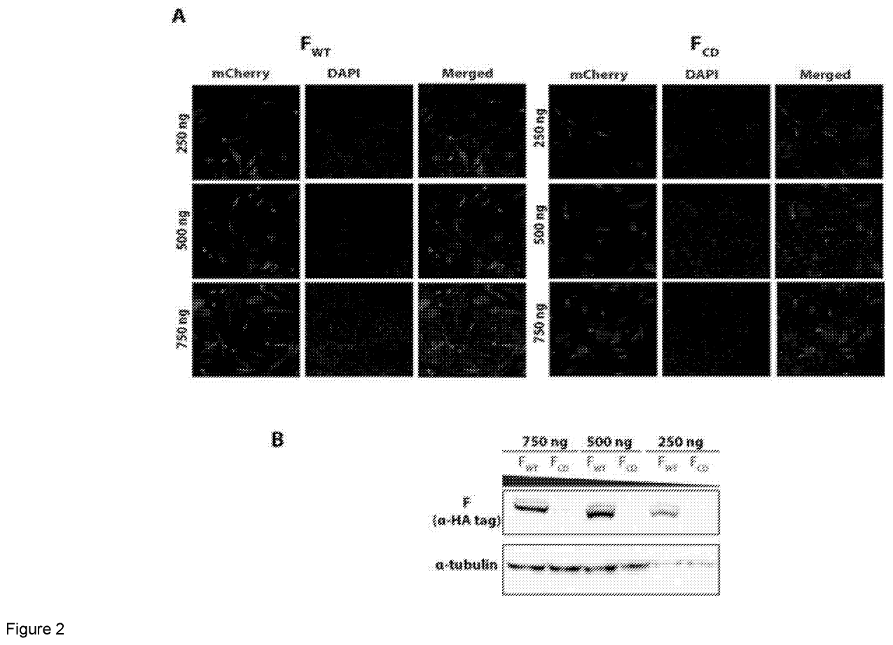

FIG. 2. Reduced expression levels of F gene in transfected cells. (A) Immunofluorescence of F_{WT} gene in transfected cells and immunofluorescence of F_{CD} gene in transfected cells in increasing concentrations. (B) Western blot of F_{WT} and F_{CD} in transfected cells with increasing concentration. Alpha tubulin was used as loading control.

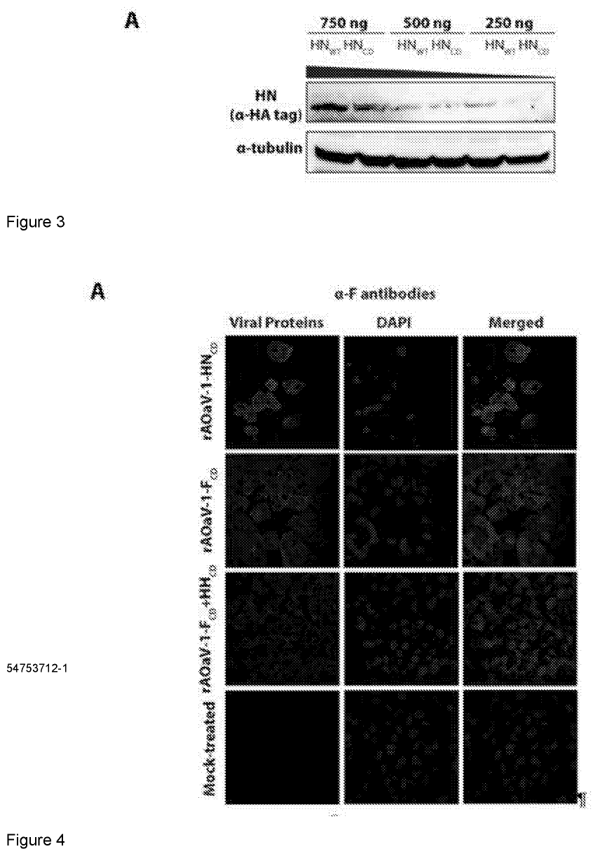

FIG. 3. Western blot expression of HN_{WT} and HN_{CD} in transfected cells with increasing concentration of 250 ng, 500 ng and 750 ng. Alpha tubulin was used as loading control.

FIG. 4. In vitro characterization of rAOaV-1. (A) Immunofluorescence of rAOaV-1-F_{CD}, rAOaV-1-HN_{CD} and rAOaV-1-F_{CD}+HN_{CD} with antibodies against F protein.

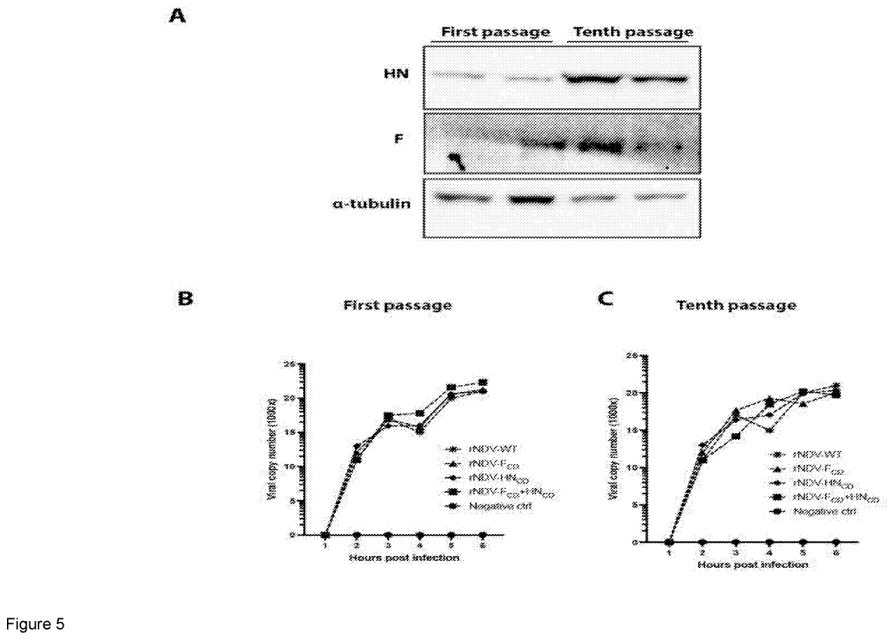

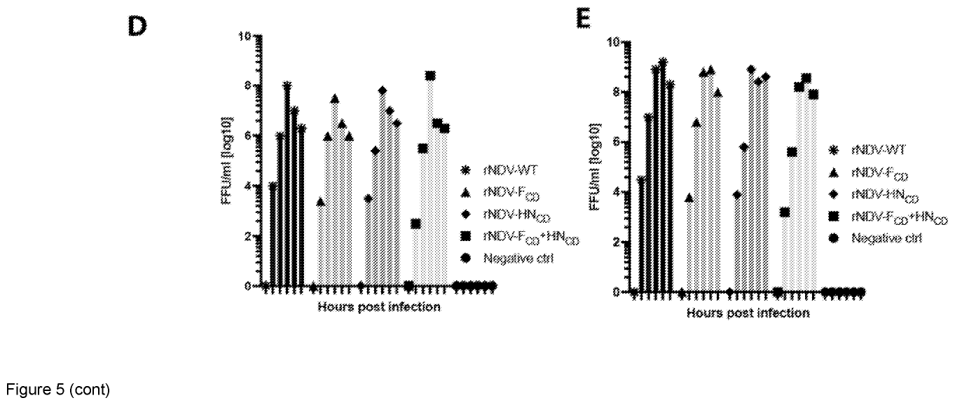

FIG. 5. In ovo attenuation of rAOaV-1 in chicken eggs. (A) The replication of rAOaV-1-F_{CD}+HN_{CD} replication after first and tenth passage in embryonated chicken eggs. (B and C) The replication kinetics by RT-PCR show a comparable replication of rAOaV-1-F_{WT}, rAOaV-1-F_{CD}, rAOaV-1-HN_{CD} and rAOaV-1-F_{CD}+HN_{CD} at first (B) and tenth (C) passage. (D and E) All recombinant viruses (rAOaV-1-F_{WT}, rAOaV-1-F_{CD}, rAOaV-1-HN_{CD} and rAOaV-1-F_{CD}+HN_{CD} showed similar replication profile by plaque assays (PFU/ml) at first (D) and tenth (E) passage.

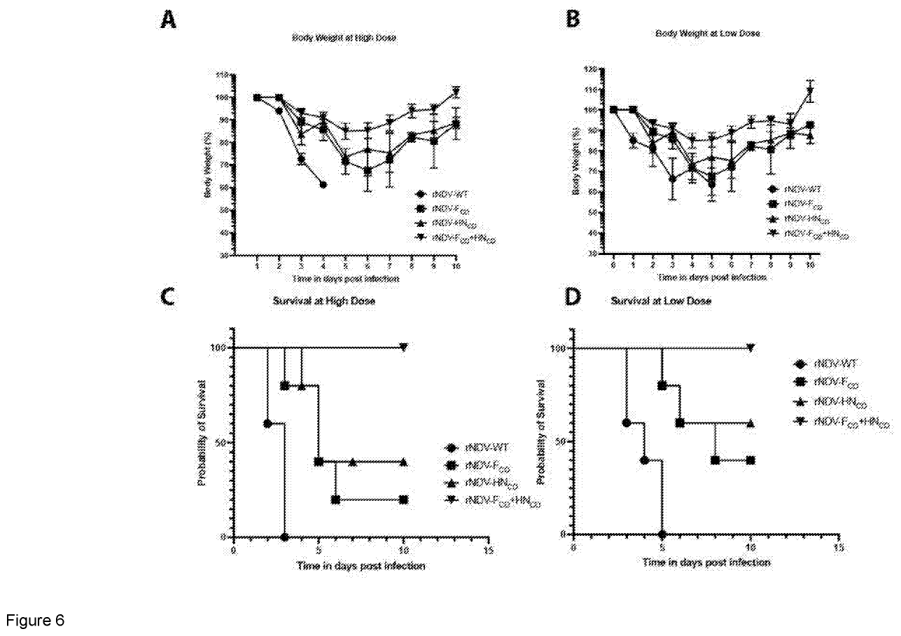

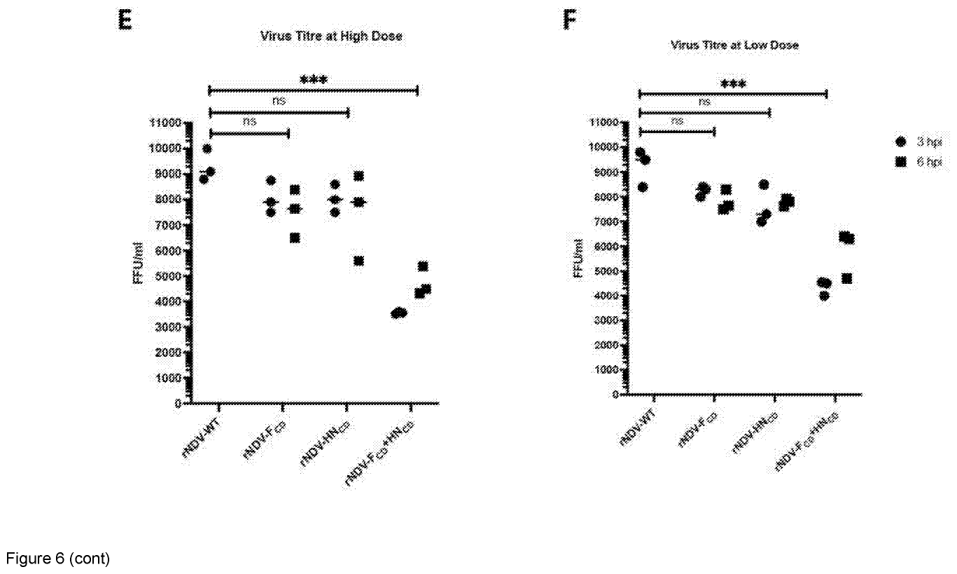

FIG. 6. Attenuation of rAOaV-1 in chicken. (A ans B) A low weight loss was noticed in chicken infected with rAOaV-1-F_{CD}+HN_{CD} compared to individual or no codon deoptimized genes containing recombinant viruses (rAOaV-1-F_{CD}, and rAOaV-1-HN_{CD}) at both high doses (A) and low doses (B). (C) The rAOaV-1-F_{CD}+HN_{CD} showed reduced mortality and appeared safe compared to rAOaV-1-F_{WT}, rAOaV-1-F_{CD}, rAOaV-1-HN_{CD}. (D) A similar trend was observed when a high dose was used. (E) Replication analysis of rAOaV-1-F_{WT}, rAOaV-1-F_{CD}, rAOaV-1-HN_{CD} and rAOaV-1-F_{CD}+HN_{CD} in chicken showed attenuated replication of rAOaV-1-F_{CD}+HN_{CD}. (F) A corresponding replication of rAOaV-1-F_{CD}+HN_{CD} was observed when higher level of infection was used.

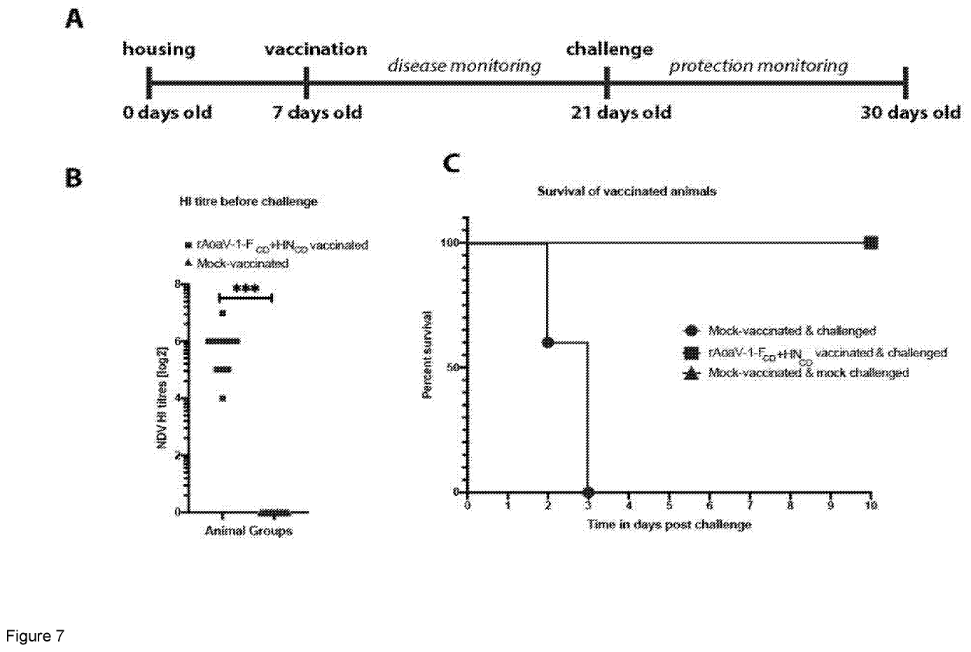

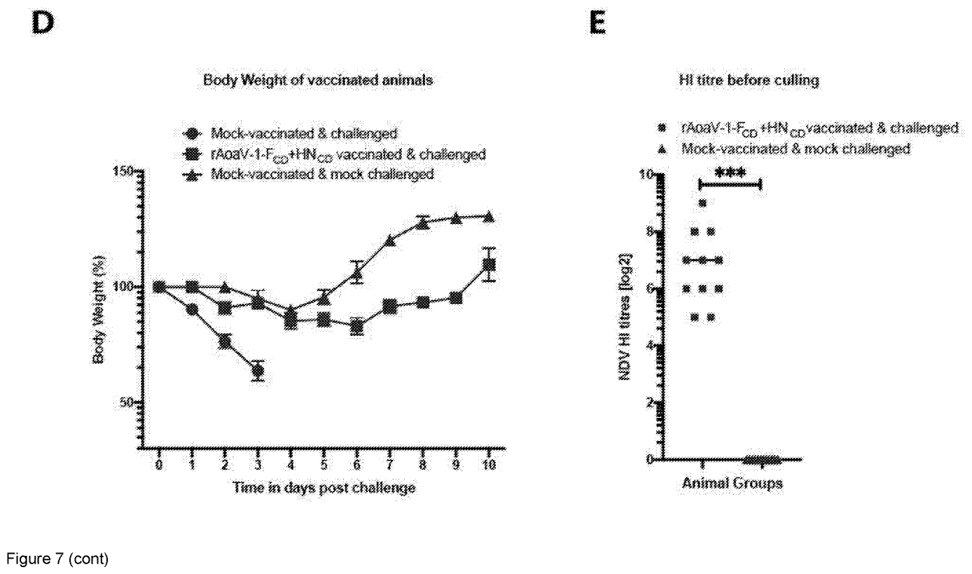

FIG. 7. Protection efficacy of recombinant rAOaV-1. (A) Experimental plan to demonstrate the protective efficacy of recombinant viruses. (B) HA titre in rAOaV-1-F_{CD}+HN_{CD} vaccinated compared to non-vaccinated animals. (C) The rAOaV-1-F_{CD}+HN_{CD} was attenuated and showed full protection against pathogenic viruses compared. (D) Correspondingly, a low weight loss was observed in animals infected with rAOaV-1-F_{CD}+HN_{CD}. (E) The HA titre before culling of animals indicate high antibodies in rAOaV-1-F_{CD}+HN_{CD} vaccinated animals compared to non-vaccinated animals.

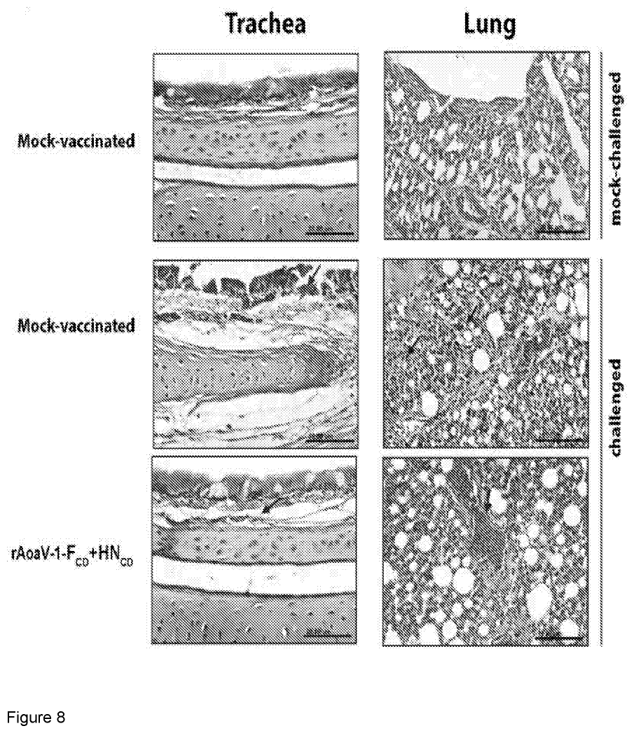

FIG. 8. A lower level of pathological changes was noticed in chicken trachea and lung infected with rAOaV-1-F_{CD}+HN_{CD} vaccinated compared to mock-vaccinated and challenged, and mock-vaccinated and mock challenged animals.

### EXAMPLE 1

In order to design and produce the codon deoptimized rAOaV-1, the F and HN genes were codon deoptimized through the SCUA algorithm described herein. The original rAOaV-1-WT nucleotide sequences were maintained all other open reading frame, other than HN and F. Runs of more than six identical nucleotides and rAOaV-1 gene-end like or gene start like sequences were removed from the computer-generated sequences by manual editing. The G/C content and the percentage of A, G, T, and C nucleotides, and of AT and GC dinucleotides, was similar between WT and codon deoptimized sequences (FIG. 1A). Percent nucleotide identity and number of nucleotide differences between WT and codon deoptimized open reading frames were less than 80%. All nucleotide changes were silent on the amino acid level. The recombinant rAOaV-1 viruses carrying individually codon deoptimized HN and F genes replicated significantly lower in chicken cells compared to rAOaV-1-WT (FIG. 1B). Notably, the recombinant rAOaV-1 carrying dual codon deoptimized HN and F genes (rAOaV-1-F_{CD}+HN_{CD}) were attenuated based on the plaque sizes compared to individually codon deoptimized rAOaV-1 (rAOaV-1-F_{CD}/rAOaV-1-HN_{CD}) or rAOaV-1-WT. The quantitative measurement of sizes of plaques in all evaluated recombinant rAOaV-1 is displayed in FIG. 1C and the difference in replication fitness is demonstrable.

Recombinant (r) rAOaV-1 were constructed using a reverse genetic system based on genotype VII strain. The rAOaV-1s were used to rescue the infectious viruses as described previously (Ayllon et al., 2013) with substantial modifications. Briefly, Vero cells were infected with modified vaccinia Ankara (MVA) expressing the T7 polymerase at a multiplicity of infection 1.0 for 6 h. These cells were transfected with Lipofectamine 2000 using rAOaVs backbones as well as supporting N, P and L gene-expression plasmids (ratio of 1:0.8:0.4:0.1) for 72 h. After 3 days post-infection, cells and cell supernatants were mixed and freeze-thawed three times at −80° C. before inoculation into 8-day-old embryonated chicken eggs. After an additional three days, individual eggs were screened using hemagglutination assay and real-time PCR as described before [OIE, 2012, Grimes, 2002, Wise et al., 2004]. Successfully rescued isolates were further propagated and purified from allantoic fluid as described previously (Kingsbury, 1966) to generate viral stock and for in vitro characterization.

### EXAMPLE 2

To determine whether the codon deoptimization of F or HN ORFs individually or in combination could lead to a reduction in protein expression levels in the absence of other viral factors, DF1 cells were transfected with 250, 500, and 750 ng of plasmid and characterized for protein expression 24 h later by epifluorescence microscopy. A reduction in the fluorescent signal and the number of fluorescent cells was observed in cells transfected with codon deoptimized constructs compared with the results for cells transfected with the rAOaV-1-WT (FIG. 2A). The effects of codon deoptimization on protein expression were also tested by Western blotting (FIG. 2B). For that, the N termini of the wt, the cd construct were fused to an HA epitope tag or GFP and used to transfect DF-1 cells. Protein expression was evaluated at 24 h post-transfection using an anti-HA Mab. The pattern of expression of both proteins correlated with that previously observed for GFP-tagged constructs. While the F-_{WT} was expressed a corresponding concentration, the F_{CD} protein was barely detected (FIG. 2B). No such difference was observed with a loading control. Similar changes were also noticed in the HN_{CD} genes compared to HN-_{WT} genes (FIG. 3). Overall, these data indicate that codon deoptimization of F or HN gene reduces protein expression, which may be attributed to differences in the percentage of codon changes introduced into the viral gene, the relative quantity of the mRNAs, or a combination thereof.

### EXAMPLE 3

Generation of recombinant codon-deoptimized rAOaV-1 viruses. Since F and HN constructs were efficiently expressed in transfected cells, we wanted to ascertain whether rAOaV-1 viruses encoding codon-deoptimized F and/or HN products could be rescued, as well as assess the effect of the deoptimization of viral F and/or HN products individually or in combination in the context of viral replication. To this end, codon-deoptimized viral F or HN RNA segments were incorporated into plasmid-based reverse genetics techniques in order to generate recombinant, codon-deoptimized viruses. We generated three different viruses containing codon-deoptimized synonymous mutations in coding regions comprising the entire F gene (rAOaV-1-F_{CD}) or entire HN (rAOaV-1-HN_{CD}) or both (rAOaV-1-F_{CD}+HN_{CD}). The identity of the recombinant viruses was confirmed by RT-PCR using restriction analysis and sequencing of the F and HN genes. Sequence data revealed that the F and HN gene in all recombinant viruses did not contain additional changes. Growth properties of codon-deoptimized recombinant rAOaV-1 were assessed in tissue culture. To analyse the replicative properties of recombinant codon-deoptimized viruses, we evaluated F expression levels in the context of viral infection by immunofluorescence (FIG. 4A).

### EXAMPLE 4

In ovo attenuation and stability of rAOaV-1. The rAOaV-1 replicate effectively in embryonated chicken eggs. All recombinant rAOaV-1 generated in this invention were propagated in chicken eggs for 10 times at least. The replication kinetics of these recombinant viruses were determined by virus quantification assays including Western blotting (FIG. 5A), RT-PCR (FIG. 5B, C) and plaque assays (FIG. 5D, E). All viruses replicated significantly and at the comparable levels indicated that codon deoptimization is stable and doesn't pose antiviral characteristics.

### EXAMPLE 5

To determine in ovo attenuation of rAOaV-1, intracerebral pathogenicity index (ICPI) was determined in 1-day-old chicks. For each ICPI test, ten 1-day-old SPF chicks were used (ten birds for test and five birds for control). The inoculum consisted of fresh, infective allantoic fluid with an HA titer for the test birds and allantoic fluid from uninfected embryonated chicken eggs for control birds. The birds were observed for clinical signs and mortality every 24 h for a period of 8 days. The scoring and determination of ICPI were done according to the method described by Alexander (1997).

In order to compare the pathogenicity of rAOaV-1-F_{CD}, rAOaV-1-HN_{CD} and rAOaV-1-F_{CD}+HN_{CD}, ICPI tests in 1-day-old chicks were performed by scoring clinical signs and mortality (Table 1). The most virulent AOaV-1 strains give indices close to 2.0, while avirulent viruses give values close to 0. In our experiments, the results of ICPI were 2 for rAOaV-1-WT, 1.18 for rAOaV-1-F_{CD} and 1.7 for rAOaV-1-HN_{CD}. The ICPI for rAOaV-1-F_{CD}+HN_{CD} was 0.0 (Table 1).

The mean death time (MDT) is hours for the minimum lethal dose to kill embryos. The minimum lethal dose is the highest virus dilution which causes all the embryos inoculated with that dilution to die. To assess MDT, 0.1 ml of the virus was inoculated into the allantoic cavity of each of five 9- to 10-day-old embryonated chicken eggs and placed in incubator at 37° C. Each egg was examined twice daily for 7 days and the times of any embryo deaths were recorded. The MDT has been used to classify rAOaV-1 strains into velogenic (taking less than 60 hours to kill), mesogenic (taking between 60-90 hours) and lentogenic (taking more than 90 hours). The MDT for rAOaV-1-WT, rAOaV-1-F_{CD}, and rAOaV-1-HN_{CD} was <60 hours. However, for rAOaV-1-F_{CD}+HN_{CD} it was >90 hours (Table 1).

The results described here show that attenuated rAOaV-1 can be used as a vaccine vector. Development of recombinant rAOaV-1 as a vaccine vector has several applications. Several foreign genes can be inserted and expressed in the same virus to obtain simultaneous immune responses to the expressed antigens in inoculated animals. For example, a single recombinant rAOaV-1 could be generated that expressed the immunogenic proteins of multiple avian pathogens or viruses of medical importance such as SARS-COV-2 (Rohaim and Munir, 2020). Alternatively, several rAOaV-1, each expressing various heterologous antigens, could be administered as a multivalent vaccine. A further extension would be to use rAOaV-1 vectors in non-avian species, where rAOaV-1 is capable of undergoing incomplete replication to the extent necessary to express inserted genes. Thus, development of rAOaV-1 as a vector should prove to be useful against avian and non-avian diseases for which suitable vaccines are not currently available.

TABLE 1
In ovo attenuation of codon deoptimized rAOaV-1

| | | Intracerebral |
|---|---|---|
| Recombinant | | pathogenicity |
| virus | Mean death time (MDT) | index (ICPI) |
| rAOaV-1-WT | <60 hours (30 hours; exact) | 2.0 |
| rAOaV-1-F_{CD} | <60 hours (38 hours; exact) | 1.8 |
| rAOaV-1-HN_{CD} | <60 hours (38 hours; exact) | 1.7 |
| rAOaV-1- | >90 hours (up to 96 hours; exact) | 0.0 |
| F_{CD} + HN_{CD} | | |

### EXAMPLE 6

In vivo characterization of codon-deoptimized viruses was assessed. We compared the virulence of rAOaV-1-WT and codon-optimized viruses in chicken. To ascertain whether the reduced expression of F, HN or both impacted the course of an in vivo virus infection, groups of chicken (n=10) were inoculated intranasally with 10⁶ or 10⁷ HA units and monitored for 10 days for signs of illness, weight loss, and mortality. As expected, codon-deoptimized viruses showed levels of attenuation and pathogenicity different from those for rAOaV-1-WT viruses. Animals infected with 10⁶ lost less body weight than infected with higher dose (10⁷) (FIG. 6A, B). In animals infected with 10⁶ or 10⁷, all animals infected with rAOaV-1-WT virus died within 4 or 5 days whereas mock infected animals survived. However, only 20% or 30% of animals infected with rAOaV-1-F_{CD} virus survived at different doses. In contrast, animals infected with the same dose of rAOaV-1-HN_{CD} virus succumbed (3% or 60%) to viral infection by day 10 (FIG. 6C, D). Interestingly, animals infected with rAOaV-1-F_{CD}+HN_{CD} viruses all survived as that of mock infected animals. We also evaluated the viral titers in lungs at high and low doses (FIG. 6E, F). Animals infected with rAOaV-1-F_{CD}+HN_{CD} showed significantly lower viral titers than animals infected with WT or individual gene codon deoptimized viruses, regardless of whether low or high dose of viruses were used. Overall, viral titres correlated with the virus dose and the degree of infection. Despite the limited attenuation observed in vitro, animals infected with rAOaV-1-F_{CD}+HN_{CD} virus showed less weight loss and mortality than animals infected with rAOaV-1-WT or rAOaV-1-F_{CD} or rAOaV-1-HN_{CD} virus.

### EXAMPLE 7

Given that rAOaV-1-F_{CD}+HN_{CD} virus was fully attenuated in animals, we hypothesized that rAOaV-1-F_{CD}+HN_{CD} virus could potentially be used as a vaccine. To evaluate this possibility, chickens were vaccinated with rAOaV-1-F_{CD}+HN_{CD} viruses or mock vaccinated with PBS. At 7 days post-vaccination (FIG. 7A), protection was evaluated by challenging vaccinated animals with virulent AOaV (FIG. 7). Vaccinated animals with rAOaV-1-F_{CD}+HN_{CD} showed high antibodies (FIG. 7B) compared to mock vaccinated animals. Only animals vaccinated rAOaV-1-F_{CD}+HN_{CD} survived challenge (FIG. 7C) while all mock-vaccinated animals drastically lost weight and died after 3-4 days of challenge (FIG. 7D). Animal vaccinated with rAOaV-1-F_{CD}+HN_{CD} showed sustained antibodies levels before culling (FIG. 7E).

### EXAMPLE 8

A significantly reduced tissue pathology was noticed in animals infected with rAOaV-1-F_{CD}+HN_{CD} viruses compared to mock-vaccinated animals (FIG. 8). These observations were observed among all organs validated by histopathological analysis exemplified here with trachea and lung. These are the important respiratory organs likely to be targeted by respiratory viruses.

The above sample embodiments should not be considered limiting to the scope of the invention whatsoever because many more embodiments and variations of embodiments are easily conceived within the teachings, scope and spirit of the instant specification.

It is to be understood that even though numerous characteristics and advantages of various embodiments of the present invention have been set forth in the foregoing description, together with the details of the structure and function of various embodiments of the invention, this disclosure is illustrative only, and changes may be made in detail, especially in matters of structure and arrangement of parts within the principles of the present invention to the full extent indicated by the broad general meaning of the terms in which the appended claims are expressed.

It will be clear that the present invention is well adapted to attain the ends and advantages mentioned as well as those inherent therein. While presently preferred embodiments have been described for purposes of this disclosure, numerous changes may be made which readily suggest themselves to those skilled in the art and which are encompassed in the spirit of the invention disclosed and as defined in the appended claims.

### REFERENCES

Krishnamurthy, S. & Samal, S. K. (1998). Nucleotide sequences of the trailer, nucleocapsid protein gene and intergenic regions of Newcastle disease virus Strain Beaudette C and completion of the entire genome sequence. Journal of General Virology 79, 2419-2424.
Phillips, R. J., Samson, A. R. & Emmerson, P. T. (1998). Nucleotide sequence of the 5′-terminus of New castle disease virus and assembly of the complete genomic sequence: agreement with the rule of six. Archives of Virology 143, 1993-2002.
de Leeuw, O. & Peeters, B. (1999). Complete nucleotide sequence of Newcastle disease virus: evidence for the existence of a new genus within the Subfamily Paramyxovirinae. Journal of General Virology 80, 131-136.
Steward, M., Vipond, I. B., Millar, N. S. & Emmer son, P. T. (1993). RNA editing in Newcastle disease virus. Journal of General Virology 74, 2539-2547.
Conzelmann, K.-K. (1996). Genetic manipulation of non-segmented negative-strand RNA viruses. Journal of General Virology 77, 381-389.
Römer-Oberdorfer, A., Mundt, E., Mebatsion, T., Buchholz, U. J. & Mettenleiter, T. C. (1999). Generation of recombinant lentogenic Newcastle disease virus from cDNA. Journal of General Virology 80, 2987-2995.
Peeters, B. P. de Leeuw, O. S., Koch, G. & Gielkens, A. L. (1999). Rescue of Newcastle disease virus from cloned cDNA: evidence that cleavability of the fusion protein is a major determinant for virulence. Journal of Virol ogy 73, 5001-5009.
Ayllon, J.; Garcia-Sastre, A.; Martínez-Sobrido, L (2013). Rescue of recombinant Newcastle disease virus from cDNA. JoVE, 80, e50830.
OIE (2012). Newcastle Disease, Biological Standards Commission, Manual of Diagnostic Tests and Vaccines for Terrestrial Animals: Mammals, Birds and Bees, 7th ed.; World Organisation for Animal Health: Paris, France, pp. 555-574.
Grimes, S. E. (2002). A Basic Laboratory Manual for the Small-Scale Production and Testing of 1-2 Newcastle Disease Vaccine; FAO Regional Office for Asia and the Pacific (RAP): Bangkok, Thailand.
Wise, M. G.; Suarez, D. L.; Seal, B. S.; Pedersen, J. C.; Senne, D. A.; King, D. J.; Kapczynski, D. R.; Spackman, E (2004). Development of a real-time reverse-transcription PCR for detection of Newcastle disease virus RNA in clinical samples. J. Clin. Microbiol. 42, 329-338.
Kingsbury, D. W. (1966). Newcastle disease virus.I. Isolation and preliminary characterization of RNA from virus particles. Journal of Molecular Biology 18, 195-203.
Coleman, J. Robert, Dimitris Papamichail, Steven Skiena, Bruce Futcher, Eckard Wimmer, and Steffen Mueller (2008). Virus Attenuation by Genome-Scale Changes in Codon Pair Bias. Science. June 27; 320 (5884): 1784-1787. doi: 10.1126/science. 1155761
Alexander, D. J. (1997). Newcastle disease and other avian Paramyxoviridae infections. In Diseases of Poultry, 10″ edition, pp. 541-569. Edited by B. W. Calnek, Iowa State University Press, Ames, lowa.
Rohaim, M, and Munir M (2020). A Scalable Topical Vectored Vaccine Candidate against SARS-COV-2. Vaccines 2020, 8 (3), 472
Weijia Wang, Xing Cheng, Paul J. Buske, JoAnn A. Suzich and Hong Jin (2019). Attenuate Newcastle disease virus by codon modification of the glycoproteins and phosphoprotein genes. Virology, 528, 144-151. DOI: https://doi.org/10.1016/j.virol.2018.12.017.

1. An attenuated velogenic avian orthoavulavirus (AOaV), wherein each of the HN and F genes of an AOaV genome of the attenuated AOaV comprises, consists essentially of, or consists of a plurality of silent mutations, as compared to a wild-type or parent AOaV from which the attenuated AOaV has been derived.

2. The attenuated AOaV according to claim 1, wherein the silent mutations have been obtained by codon deoptimsation strategies.

3. The attenuated AOaV according to claim 2, wherein the codon optimisation strategy comprises using a Smart Codon Usage Algorithm (SCUA):
$$SCUA = \frac{\frac{CfOt}{CfOr} NOt}{NOr} \times vgf$$
Here,
CfOt: frequency of codon occurrence in test sequence
CfOr: frequency of codon occurrence in reference sequence
NOt: number of codon occurrences in test sequence
NOr: number of codon occurrences in reference sequence
vgf: viral genomic features.

4. The attenuated AOaV according to either of claim 1 or 2 wherein each of the HN and F genes includes at least 5, 10, 15, 20, 25, 30, 40, 50 or more mutated codons.

5. The attenuated AOaV according to any preceding claim comprising one or more substitutions, inversions, deletions, or additions in any one or more of the NP, P, M, or L genes.

6. The attenuated AOaV according to any preceding claim, which has been further modified in order to express one or more proteins or antigenic fragments thereof, from another pathogen, such as another virus, or organism.

7. A vaccine or pharmaceutical composition comprising the attenuated AOaV according to any preceding claim, together with a pharmaceutically acceptable excipient therefor.

8. A modified AOaV genome encoding for an attenuated AOaV according to any of claims 1-5.

9. A vector comprising the modified AOaV genome according to claim 7.

10. The attenuated AOaV, vaccine or pharmaceutical composition, modified AOaV genome or vector according to any preceding claim, for use in therapy.
